(19) United States
(12) Patent Application Publication
(10) Pub. No.: US 2023/0200625 A1
(43) Pub. Date: Jun. 29, 2023

(54) SYSTEMS AND METHODS OF COMPUTER-ASSISTED LANDMARK OR FIDUCIAL PLACEMENT IN VIDEOS

(71) Applicant: KALIBER LABS INC., San Francisco, CA (US)

(72) Inventors: Bipul KUMAR, San Francisco, CA (US); Mathew FRANCIS, San Francisco, CA (US); Gaurav YADAV, San Francisco, CA (US); Biswajit Dev SARMA, San Francisco, CA (US); Tim BAKHISHEV, San Francisco, CA (US); Chandra JONELAGADDA, San Francisco, CA (US); Mark RUIZ, San Francisco, CA (US); Ray RAHMAN, San Francisco, CA (US)

(21) Appl. No.: 17/996,212

(22) PCT Filed: Apr. 13, 2021
(86) PCT No.: PCT/US2021/027000

§ 371 Date: Oct. 13, 2022

Related U.S. Application Data
(60) Provisional application No. 63/030,721, filed on May 27, 2020.
(60) Provisional application No. 63/143,380, filed on Jan. 29, 2021.

(30) Foreign Application Priority Data
Apr. 13, 2020 (IN) 202041015993

(51) Int. Cl. A61B 1/00; G06T 19/20; G06T 7/73; A61B 1/317

(52) U.S. Cl. CPC A61B 1/0005; G06T 19/20; G06T 7/74; A61B 1/317; A61B 1/00009; G06T 2219/2004; G06T 2210/41; G06T 2207/30204; G06T 2207/30196

(57) ABSTRACT

Various embodiments of the invention provide systems and methods to assist or guide an arthroscopic surgery (e.g., surgery of the shoulder, knee or hip) or other surgical procedure by the placement of arbitrary landmarks in one or more locations in surgical field of view. The systems and methods comprise steps of receiving a video stream from an arthroscopic or other imaging device; receiving one or more sets of coordinates of one or more landmarks; overlaying the one or more landmarks on the video stream; and displaying the overlay on one or more display devices intraoperatively so as to be used by an operator during the arthroscopic or other medical procedure.

### CROSS-REFERENCE TO RELATED APPLICATIONS

This PCT application claims priority to Indian Provisional Patent Application No. 202041015993, filed Apr. 13, 2020, and U.S. Provisional Application Nos. 63/030,721, filed May 27, 2020, and 63/143,380, filed Jan. 29, 2021, the contents of all of which are fully incorporated herein by reference.

### BACKGROUND

Field of the Invention: Embodiments of the invention relate to systems, devices, and methods to assist surgical procedures, particularly using Artificial Intelligence (AI).

In recent years, Artificial Intelligence has begun to be developed to be used to process images to recognize features of a human face as well as different anatomical structures in a human body. These AI tools can be used to automatically recognize an anatomical feature to assist an operator during a medical procedure. Computational methods such as machine learning and deep learning algorithms can be used for image or language processing to gather and process information generated in a medical procedure. The hope is to use AI algorithms that can then be used to or improve the outcome of the surgery. Current AI-assisted surgical systems and methods are still less than ideal in many respects to be used to, for example, guide a surgical procedure. Accordingly, improved AI-assisted surgical systems and methods are desired.

### BRIEF DESCRIPTION OF THE INVENTION

Various embodiments of the invention relate to computer-implemented medical systems, devices, and methods to guide a surgical procedure such as by identifying and labelling anatomical features in real-time and placing one or more landmarks on the identified anatomical feature. Surgeons use physical landmarks to underpin a variety of cognitive tasks; keeping track of latent vascularity, staple lines, suture locations, latent anatomical structures, etc. The landmarks are typically placed using dyes, cauterization marks, etc. In some embodiments, needles are inserted from the outside to mark points. Placing a physical landmark which may require implanting an object in the patient's body may add to complications of the surgery and physically inhibit movement of surgical tools in the course of surgery. Other issues may involve a mistake made by an operator in the course of a surgery which can be costly. For example, it may be difficult or impossible for an operator to know the exact location of a critical anatomical feature that is hidden from a camera (e.g., a camera used during an arthroscopic or endoscopic surgery), or a change of field of view may make it difficult for the operator to identify the location of a landmark. Therefore, computer-implemented medical systems, devices, and methods such as Artificial Intelligence (AI) tools, particularly for guiding medical procedures by applying a virtual landmark on the patient's body (e.g., on an organ or anatomical feature) can be valuable. These AI tools can have their limitations in accurately and reliably predicting a tool, anatomical structure, or detecting a procedure. In a fast-paced surgical procedure, the AI tool may need to also make predictions with low latency to provide real time assistance to an operator.

Recognized herein is the need for fast, accurate and reliable AI tools to assist an operator in real time during the course of a surgical operation or other medical procedure by placing a virtual landmark on a location of interest to facilitate the surgical (or other medical) procedure for the operator (e.g., surgeon, interventional radiologist) and to improve an outcome of the surgery or other medical procedure. Accordingly, various aspects and embodiments of the present invention provide a pipeline of machine learning algorithms that is versatile and well trained for unique needs of landmarks in various medical procedures.

Various embodiments of the invention described herein provides systems, devices, and methods that can receive information (e.g., image, voice, user inputs) prior to and during a medical procedure (e.g., a surgery), process the received information to identify features associated with placing a landmark associated with the procedure, and place a virtual landmark at a location of interest in real time during the procedure.

Aspects of the present invention also aid surgeons to place a landmark on a location of interest intraoperatively by using images acquired preoperatively using imaging modalities and associated methods such as fluoroscopy, magnetic resonance imaging (MRI), or computed tomography (CT) scanning. In one or more embodiments, the preoperative images can be that of a surgical field of view and Artificial Intelligence (AI) can be applied to preoperatively generated images to overlay the images and/or location of a landmark onto a real-time video stream of a surgical procedure to provide guidance to a surgeon. We refer to AI modules/algorithms used intraoperatively, preoperatively, or postoperatively to assist with the surgical procedure or improve an outcome of the procedure as Surgical AI.

One aspect of the present invention provides systems for assisting an arthroscopic procedure such as a repair to a shoulder, knee, hip, ankle or other joint by allowing computer-implemented arbitrary landmark placement, the system comprising one or more computer processors and one or more non-transitory computer-readable storage media storing instructions that are operable, when executed by the one or more computer processors, to cause the one or more computer processors to perform operations comprising: receiving a video stream from an arthroscopic imaging device; receiving one or more sets of coordinates of one or more landmarks; overlaying the one or more landmarks on the video stream; and displaying the overlay on one or more display devices intraoperatively to be used by an operator during the arthroscopic procedure. Application of embodiments of the system to the assistance to other medical procedures (e.g., by the placement of arbitrary landmarks) including minimally invasive procedures such as endoscopic, laparoscopic, and interventional cardiovascular procedures is also contemplated. Examples of such minimally invasive procedures can include one or more of Gastro-intestinal (GI) procedures (e.g., biopsy of the intestines, removal of polyps, bariatric surgery, stomach stapling/vertical banded gastroplasty), urological procedures (e.g., removal of kidney stone, bladder repair), gynecological procedures (e.g., a dnc, removal of uterine fibroids) and laparoscopic procedures (e.g., an appendectomy, cholecystectomy, colectomy, hernia repair, nissen fundoplication).

In some embodiments, the operations further comprise identifying and labeling one or more elements in the video stream using at least one of a trained computer algorithm. In some embodiments, the one or more elements comprise one or more of an anatomical structure, a surgical tool, an operational procedure or action, or a pathology. In some embodiments, the identifying and labeling the one or more elements in the video stream comprises using one or more software modules (herein modules). In some embodiments, the one or more modules may comprise modules for performing video stream decomposition, tool recognition, anatomy recognition, tool tracking, gesture recognition, landmark point registration, or anatomy and landmark tracking. In some embodiments, the system recommends one or more landmarks based at least partially on the identified elements.

In some embodiments, the operations further comprise: storing the one or more sets of coordinates of one or more landmarks; changing a view of the display to omit the overlaid landmark from being displayed; reverting the view to the previous display; identifying the one or more set of coordinates for the one or more landmarks; and re-overlaying the one or more landmarks. In some embodiments, the operator activates the changing and the reverting steps. In some embodiments, changing a view step is activated automatically based on a change in an identified anatomical structure or pathology.

In some embodiments, the one or more sets of coordinates of the one or more landmarks is provided by an operator (e.g., a surgeon, interventional cardiologist, radiologists, etc.) intraoperatively. In some embodiments, the one or more sets of coordinates of the one or more landmarks is provided by an operator preoperatively. In some embodiments, the one or more sets of coordinates of the one or more landmarks is generated from one or more medical images of a subject. In some embodiments, the one or more medical images are radiological images of the subject. In some embodiments, the radiological images are from a joint other boney structure of the subject. In some embodiments, the radiological images are associated with a shoulder, a knee, a hip, ankle or elbow of the subject. In some embodiments, the radiological images are generated using fluoroscopy, magnetic resonance imaging (MRI), computed tomography (CT) scanning, positron emission tomography (PET) scanning or ultrasound imaging.

In some embodiments, the video stream is provided by an arthroscope (or other imaging device) during the arthroscopic procedure. In various embodiments, the arthroscopic procedure may correspond to one or more of the following types of procedures (for which embodiments of the systems and modules may be so configured for assisting with): ACL repair in a knee surgery; graft placement procedure, e.g., that used in a superior capsule reconstruction of a torn rotator cuff; a decompression procedure; a removal of or a resection of one or more inflamed tissues; removal of or a resection of one or more frayed tendons where the video stream is monocular. In one or more of the above and other procedures the video stream may be stereoscopic or monocular unless otherwise noted in the specific procedure. Also in various implementations embodiments of the systems of the invention can be configured to toggle or switch back and forth between monocular or stereoscopic inputted video stream and associated outputted video overlays.

In some embodiments, the one or more computer processors receive the video stream from one or more camera control units using a wired media connection. In some embodiments, a latency between receiving the input from the digital camera and overlay the output and the videos stream is at most 40 milliseconds (ms) to accommodate a digital camera with about 24 frames per second (fps). In some embodiments, the latency between receiving the input from the digital camera and overlay the output and the videos stream is no more than a time between two consecutive frames from the digital camera.

In some embodiments, the one or more computer processors receive the video stream from one or more camera control units using a network connection. In some embodiments, the interventional imaging device is a digital camera specialized for arthroscopic use. In some embodiments, the digital camera is mounted on a rigid scope, suitable for work in the arthroscopic joints. In some embodiments, the camera control unit is configured to control a light source, capture digital information produced by the digital camera. In some embodiments, the camera control unit converts the digital information produced by the digital camera into the video stream. In some embodiments, the camera control unit record the digital information produced by the digital camera in a memory device. In some embodiments, the memory device is a local memory device while in others it may be a cloud-based memory device. In some embodiments, the digital camera is connected to a camera control unit which in various embodiments may be configured to overlay the output from with the one or more computer processors with the video stream.

In some embodiments, the system further comprises a display monitor. In some embodiments, the one or more computer processors comprise a central processing unit or a Graphical Processing Unit (also referred to a s GPU). In some embodiments, the system further comprises a mechanism to receive an input from the at least one operator (to activate or stop marking the landmark) intraoperatively. In various embodiments, the mechanism is configured to receive the input via one or more of a push-button, a touchscreen device, a pointing device, (e.g., a mouse or a head mounted pointing device), a foot pedal, a gesture recognition system, or a voice recognition system. In some embodiments, the one or more landmarks are tracked during the arthroscopic or other medical procedure. In some embodiments, the tracking of one or more landmarks is associated with the set of coordinates of the one or more landmarks relative to at least one or more of an anatomical structure, a injury or pathology or the structure, an implant placed in the structure or a repair of the structure.

In some embodiments, the displaying the one or more landmarks are overlaid on the displaying video stream. In some embodiments, the one or more landmarks are displayed as the relative anatomical structure is identified in the video stream. In some embodiments, the operator can select to render the one or more landmarks temporarily invisible or throughout the arthroscopic or other medical procedure.

Another aspect of the invention provides systems for assisting an arthroscopic or other medical procedure by allowing computer-implemented arbitrary landmark placement using radiological imaging, the system comprising one or more computer processors and one or more non-transitory computer-readable storage media storing instructions that are operable, when executed by the one or more computer processors, to cause the one or more computer processors to perform operations. In some embodiments, the operations comprise: receiving at least one radiological image of a subject; identifying one or more anatomical features in the at least one radiological image with a trained machine learning algorithm; generating a 3D representation of the identified anatomical features; receiving a location of one or more landmarks from an operator; overlaying the one or more landmarks on the 3D representation of the anatomical structures; and displaying the overlay on a displaying device to be used by the operator. Again Application of embodiments of the above system to the assistance of other medical procedures including minimally invasive procedures such as endoscopic, laparoscopic, and interventional cardiovascular procedures is also contemplated.

In some embodiments, the anatomical features comprise a bony structure or a tendon. In some embodiments, the at least one radiological image comprises one or more of an MRI scan, a CT scan, a PET scan, an ultrasound image or a combination thereof. In some embodiments, the at least one radiological image includes an image of a landmark. In some embodiments, the operations further comprise identifying the location of the landmark. In some embodiments, the operations further comprise recommending a location for a landmark based at least in part on the identified location of the landmark in at least one radiological image.

In some embodiments, the at least one radiological image or the one or more landmarks are blended with a video stream from an imaging device. In some embodiments, the blended image is displayed on a displaying device. In some embodiments, the displaying of the blended image occurs during the arthroscopic or other medical procedure. In some embodiments, the imaging device is an interventional imaging device such as an ultrasound imaging device or a fluoroscopic imaging device. In various embodiments, the video stream may be monocular or stereoscopic and the system can be configured recognize either to toggle back and for between either type and generate the associated output accordingly.

Another aspect of the current invention provides computer-implemented methods for assisting an arthroscopic or other medical procedure. In some embodiments, the methods comprise: receiving a video stream from an imaging device; receiving one or more sets of coordinates of one or more landmarks; overlaying the one or more landmarks on the video stream; and displaying the overlay on one or more display devices intraoperatively to be used by an operator during the arthroscopic or other medical procedure. Application of embodiments of the above methods to the assistance of other medical procedures including minimally invasive procedures such as endoscopic, laparoscopic, and interventional cardiovascular procedures is also contemplated.

In some embodiments, the method further comprises identifying and labeling one or more elements in the video stream using at least one of a trained computer algorithm, where the one or more elements comprise one or more of an anatomical structure, a surgical tool, an operational procedure or action, or a pathology. In some embodiments, identifying and labeling the one or more elements in the video stream comprises using one or more modules. In some embodiments, the one or more modules comprise one or more modules for video stream decomposition, tool recognition, anatomy recognition, tool tracking, gesture recognition, landmark point registration, or anatomy and landmark tracking. In some embodiments, the one or more landmarks are recommended based at least partially on the identified elements.

In some embodiments, the method further comprises: storing the one or more sets of coordinates of one or more landmarks; changing a view of the display to omit the overlaid landmark from being displayed; reverting the view to the previous display; identifying the one or more set of coordinates for the one or more landmarks; and re-overlaying the one or more landmarks. In some embodiments, the operator activates the changing and the reverting steps. In some embodiments, the changing a view step is activated automatically based on a change in an identified anatomical structure or pathology.

In some embodiments, the one or more sets of coordinates of the one or more landmarks is provided by an operator intraoperatively. In some embodiments, the one or more sets of coordinates of the one or more landmarks is provided by an operator preoperatively. In some embodiments, the one or more sets of coordinates of the one or more landmarks is generated from one or more medical images of a subject. In some embodiments, the one or more medical images are radiological images. In some embodiments, the radiological images are generated using fluoroscopy, MRI, or CT scanning. In some embodiments, the video stream is provided by an arthroscope during an arthroscopic procedure. In some embodiments, the arthroscopic procedure is used in a rotator cuff implant surgery. In some embodiments, the arthroscopic procedure is used in an ACL tunnel placement in a knee surgery. In some embodiments, the video stream is monocular. In some embodiments, the video stream is stereoscopic.

In some embodiments, the receiving the one or more video stream from the digital camera is performed using a wired media connection. In some embodiments, a latency between receiving the input from the digital camera and displaying an overlay of the output and the videos stream is at most 40 milliseconds (ms) to accommodate a digital camera with about 24 frames per second (fps). In some embodiments, the latency between receiving the input from the digital camera and overlay the output and the videos stream is no more than a time between two consecutive frames from the digital camera. where the receiving the one or more video stream from the digital camera is performed using a network connection.

In some embodiments, the method is performed using one or more computer processing units. In some embodiments, the one or more computer processing units comprise a central processing unit or a Graphical Processing Unit. In some embodiments, the interventional imaging device is a digital camera. In some embodiments, the digital camera is mounted on a scope.

In some embodiments, the camera control unit is configured to control a light source and capture digital information produced by the digital camera. In some embodiments, the camera control unit is configured to convert the digital information produced by the digital camera into the video stream.

In some embodiments, the camera control unit records the digital information produced by the digital camera in a memory device which may be a local memory device resident or operatively coupled to a computer system that performs one on more operations/steps of the method or remote memory device such as a cloud-based memory device. In some embodiments, the digital camera is connected to a camera control unit. In some embodiments, the video stream is received from the camera control unit by the one or more computer processing units to be processed. In some embodiments, the camera control unit is configured to overlay the output from the process by one or more computer processing units onto the video stream. In some embodiments, the method further comprises a display monitor.

In some embodiments, the method further comprises utilizing a mechanism to receive an input from the at least one operator to activate or stop marking the landmark intraoperatively. In one or more embodiments, the mechanism may be configured to receive the input via a push-button, a touchscreen device, a foot pedal, a gesture recognition method, or a voice recognition method.

In some embodiments, the one or more landmarks are tracked during the arthroscopic or other medical procedure (endoscopic, laparoscopic, cardioscopic procedure). In some embodiments, the tracking of one or more landmarks is associated with the set of coordinates of the one or more landmarks relative to at least one of an anatomical structure, an injury or pathology of the structure, an implant in the structure or a repair of the structure. In some embodiments, the display of the one or more landmarks is blended with the displaying the video stream. In some embodiments, the one or more landmarks are displayed as the relative anatomical structure is identified in the video streaming. In some embodiments, the operator can select to render the one or more landmarks invisible temporarily or throughout the arthroscopic procedure.

Another aspect of the present invention provides computer-implemented methods for assisting an arthroscopic or other medical procedure by arbitrary landmark placement using radiological imaging. In some embodiments, the methods comprise: receiving at least one radiological image of a subject; identifying one or more anatomical features in the at least one radiological image a trained machine learning algorithm; generating a 3D representation of the identified one or more anatomical features; receiving a location of one or more landmarks from an operator; overlaying the one or more landmarks on the 3D representation of one or more anatomical features; and displaying the overlay on a displaying device to be used by the operator. Application of embodiments of the methods to the assistance of other medical procedures including minimally invasive procedures such as endoscopic, laparoscopic, and interventional cardiovascular procedures is also contemplated.

In some embodiments, the anatomical features comprise a bony structure or a tendon. In some embodiments, the at least one radiological image comprises one or more of an MRI scan, a CT scan, or a combination thereof. In some embodiments, the at least one radiological image includes an image of a landmark. In some embodiments, the method further comprises identifying the location of the landmark.

In some embodiments, the method further comprises recommending a location for a landmark based at least in part on the identified location of the landmark in at least one radiological image. In some embodiments, the at least one radiological image or the one or more landmarks are overlaid on the video stream from an imaging device. In some embodiments, the blended image is displayed on a displaying device. In some embodiments, the displaying of the blended image is during an arthroscopic procedure. In some embodiments, the imaging device is an interventional imaging device. In some embodiments, the video stream is monocular. In some embodiments, the video stream is stereoscopic.

Another aspect of the present invention provides a non-transitory computer readable medium comprising machine executable code that, upon execution by one or more computer processors, implements any of the methods above or elsewhere herein.

Another aspect of the present invention provides a system comprising one or more computer processors and computer memory coupled thereto. The computer memory comprises machine executable code that, upon execution by the one or more computer processors, implements any of the methods above or elsewhere herein.

Additional aspects and advantages of the present invention will become readily apparent to those skilled in this art from the following detailed description, wherein only illustrative embodiments of the present invention are shown and described. As will be realized, the present invention is capable of other and different embodiments, and its several details are capable of modifications in various obvious respects, all without departing from the invention. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematic example of a hardware configuration of a system for assisting an arthroscopic procedure by allowing computer-implemented arbitrary landmark placement, according to some embodiments.

FIGS. 2A-2B show examples of landmark placement on a model femoral condyle, according to some embodiments.

FIG. 3 shows a schematic of an exemplary flow chart of a landmark placement system, according to some embodiments.

FIG. 4 shows a schematic of an exemplary workflow of landmark placement using a preoperative image, according to some embodiments.

FIG. 5 shows a schematic of an exemplary workflow of a system to recommend a landmark placement, according to some embodiments.

FIG. 6 shows a schematic flowchart of an exemplary system to process a stereoscopic video stream, according to some embodiments.

FIG. 7 shows a computer system that is programmed or otherwise configured to implement methods provided herein, according to some embodiments.

FIG. 8 shows an example of placing a landmark and compensating for an occlusions, according to some embodiments.

FIG. 9 shows an example of a landmark being cleared from an object, according to some embodiments.

FIG. 10A-10B show an example of stabilizing a landmark against a movement of a camera with respect to an anatomical structure, according to some embodiments.

FIG. 11A-11B show an example of feature detection, according to some embodiments.

### DETAILED DESCRIPTION

While various embodiments of the present invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Various embodiments of the invention provide computer-implemented medical systems, devices, and methods for assisting surgeons in an intraoperative setting using AI. The systems, devices, and methods disclosed herein may improve upon existing methods of surgical landmark placement by providing a fast and reliable classification (e.g., real-time) of various elements involved in a surgical operation (e.g., surgical tools, anatomical features, operation procedures) and placement of a virtual landmark with high precision and accuracy based on the classification of various elements. For example, systems, devices, and methods provided herein may use AI methods (e.g., machine learning, deep learning) to build a classifier which improves a real-time classification of elements involved in a surgical operation and identifies a location of a landmark by intraoperative command from an operator (e.g., using a surgical probe) or by processing preoperative medical images (e.g., on MRI, CT scan, or fluoroscopy), where the preoperative medical images contains a landmark. An AI approach may leverage large datasets in order gain new insights from the datasets. The classifier model may improve real-time characterization of various elements involved in an operation which may lead to higher operation success rate. The classifier model may provide an operator (e.g., surgeon, operating room nurse, surgical technician) with information for more accurate placement of a virtual landmark which eliminates the shortcomings of a physical landmark. The virtual landmark is trackable, removable, or changeable by using a button. The virtual landmark may not inhibit physical movement of the surgical tools during the surgery. The systems and methods here can overlay a landmark on a video stream of the surgery on demand (e.g., to show or not display based on a command from an operator).

Reference will now be made in detail to various embodiments, examples of which are illustrated in the accompanying drawings. In the following detailed description, numerous specific details are set forth in order to provide a thorough understanding of the present invention and the described embodiments. However, the embodiments of the present invention are optionally practiced without these specific details. In other instances, well-known methods, procedures, components, and circuits have not been described in detail so as not to unnecessarily obscure aspects of the embodiments. In the drawings, like reference numbers designate like or similar steps or components.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items.

As used herein, the term “if” is optionally construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” is optionally construed to mean “upon determining” or “in response to determining” or “in accordance with a determination” or “upon detecting” or “in response to detecting” that the stated condition precedent is true, depending on the context.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, the terms “comprises,” “comprising,” or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, the terms “subject” and “patient” are used interchangeably. As used herein, the terms “subject” and “subjects” refers to a human being. In certain embodiments, the subject is going through a surgical operation. In certain embodiments, the subject is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “surgical AI” or “surgical AI module”, as used herein, generally refer to a system, device, or method that uses Artificial Intelligence algorithms to assist before, during, and/or after a surgical operation. A surgical AI module can be defined as a combination of input data, machine learning or deep learning algorithms, training datasets, or other datasets.

The term “machine learning”, as used herein, may generally refer to computer algorithms that can improve automatically over time. Any description herein of machine learning can be applied to Artificial Intelligence, and vice versa, or any combination thereof.

As used herein, the terms “continuous,” “continuously” or any other variation thereof, generally refer to a substantially uninterrupted process or a process with time delay that is acceptable in the context of the process.

The terms “video stream”, or “video feed”, as used herein, refer to data generated by a digital camera. Video feed may be a sequence of static or moving pictures.

The terms “region,” “organ,” “tissue,” “structure”, as used herein, may generally refer to anatomical features of the human body. A region may be larger than an organ and may comprise an organ. An organ may comprise one or more tissue types and structures. A Tissue may refer to a group of cells structurally joined to complete a common function. A structure can refer to a part of a tissue. In some embodiments, a structure may refer to one or more parts of one or more tissues joined together to create an anatomical feature.

The terms “surgical field of view,” or “field of view,” as used herein, refer to the extent of visibility captured by an interventional imaging device. Field of view may refer to the extent of visual data captured by a digital camera that is observable by human eye.

The term “decision,” as described herein, may refer to outputs from a machine learning or AI algorithm. A decision may comprise labeling, classification, prediction, etc.

The term “interventional imaging device,” as used herein, generally refers to an imaging device used for medical purposes. The interventional imaging device may refer to an imaging device that is used in a surgical operation e.g. one or more of an arthroscope, cardioscope, endoscope or laparoscope other like device. The surgical operation, in some embodiments, may be a simulation of an operation or other medical procedure.

The term “operator,” used herein, refers to a medical professional involved in a surgical operation. An operator can be a surgeon, an operating room nurse, a surgical technician.

The term “landmark”, “arbitrary landmark”, “virtual landmark”, and “fiducial marker” are as used interchangeably herein to refer to marks used to guide surgical or other medical procedures.

One aspect of the invention provides a system for assisting an arthroscopic procedure by allowing computer-implemented arbitrary landmark placement. The system may comprise one or more computer processors and one or more non-transitory computer-readable storage media storing instructions that are operable, when executed by the one or more computer processors, to cause the one or more computer processors to perform operations comprising: receiving a video stream from an arthroscopic imaging device; receiving one or more sets of coordinates of one or more landmarks; overlaying the one or more landmarks on the video stream; and displaying the overlay on one or more display devices intraoperatively to be used by an operator during the arthroscopic procedure. In some embodiments, an operator (e.g., a surgeon) provides the one or more sets of coordinates of one or more landmarks preoperatively.

The video stream may be provided by the arthroscopic imaging device during the arthroscopic procedure. In some embodiments, the arthroscopic imaging device comprises a digital camera. The video stream may be obtained from a digital camera specialized for an arthroscopic procedure. The digital camera may be mounted on a rigid scope, suitable for work in the arthroscopic joints. The scope may comprise an optical fiber which illuminates a field of view of the surgery. The digital camera may be mounted to a camera control unit. In some embodiments, a camera control unit is configured to capture digital information produced by the digital camera. In some embodiments, the camera control unit converts the digital information produced by the digital camera into the video stream. In some embodiments, the camera control unit is configured to control a light source. In some embodiments, the camera control unit is configured to record the digital information produced by the digital camera in a memory device. In some embodiments, the memory device used to record the digital information is a local memory device. In some embodiments, the camera control unit is configured to overlay the output from the one or more computer processors with the video stream. In some embodiments, the memory device is a remote or cloud-based memory device. The camera control unit may send the video stream to the one or more computer processors. In some embodiments, there is more than one camera control units. In some embodiments, there are 2, 3, 4, 5, or more camera control units. The one or more camera control units may send the video streams to the computer processors via network connection or a wired media connection. The video stream may be stereoscopic or monocular. In some embodiments, the system further comprises a display monitor. In some embodiments, the system comprises a mechanism to receive an input from the at least one operator (e.g., to activate or stop marking the landmark) intraoperatively. In some embodiments, the mechanism receives the input via a push-button, a touchscreen device, a foot pedal, a gesture recognition system, or a voice recognition system.

In some embodiments, the one or more sets of coordinates are provided during the surgery using a digital pointer (e.g., a computer mouse or related device) that can mark an image in the video stream to select a point or a region for performing surgery and/or a surgical action (e.g., tissue resection, ablation, etc.). In some embodiments, an operator (e.g., a surgeon) provides the one or more sets of coordinates intraoperatively by indicating the desired location using a standard surgical probe. In some embodiments, after the coordinates of a desired location are selected or indicated, an operator can issue a command so the system can register the location. In some embodiments, the system receives the register command from the operator via a push-button, a touchscreen device, a foot pedal, a gesture recognition system, or a voice recognition system.

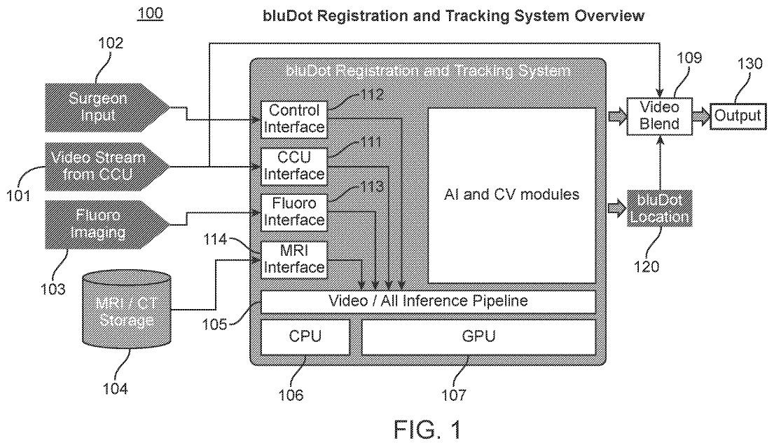

FIG. 1 shows a schematic example of a hardware configuration of the system described herein. The exemplary system 100 may comprise a plurality of inputs. The plurality of inputs may comprise a video stream input 101, an operator (e.g., surgeon) input 102, and one or more preoperative imaging inputs. The preoperative imaging inputs may comprise a fluoroscopy imaging input 103, a medical data system (e.g., radiology imaging such as MRI, or CT scan) input 104. In some embodiments, each of the plurality of inputs is connected to a corresponding interface. For example, video stream input 101 may be connected to a camera control unit (CCU) 111, operator input 102 may be connected to a control interface 112, fluoroscopy imaging input 103 is connected to a fluoroscopy interface 113, or medical data system input 104 may be connected to a medical data system (e.g., radiology imaging) interface 114. Each of the interfaces may be configured to receive an input from their corresponding inputs. The system 100 may support other external interfaces to receive input in various modalities from the surgeon, clinical data systems, surgical equipment, etc. The plurality of inputs received by a plurality of interfaces may then be sent to a processing unit to be processed using an artificial intelligence (AI) pipeline. In some embodiments, the processing unit may comprise a central processing unit (CPU) 106, a graphical processing unit (GPU) 107, or both. In some embodiments, a CPU or a GPU comprises a plurality of CPUs or GPUs. The CPU or GPU may be connected to the plurality of interfaces via a media connector (e.g., an HDMI cable, a DVI connector). The CPU or GPU may be connected to the plurality of interfaces (e.g., surgical video camera) over network connection (e.g., TCP/IP), which may provide more flexibility with less wired connections. In some embodiments, the latency in video processing and playback may be higher when the connection is via network as compared to a media connector. In some embodiments, the network connection may be a local network connection. The local network may be isolated including a set of predefined devices (e.g., devices being used in the surgery.) Lower latency may be more desirable for real-time feedback (e.g., during a surgery). In some embodiments, a system setup with higher latency can be used for training purposes (e.g., a mock surgery). The AI pipeline may comprise one or more machine learning modules or AI modules comprising one or more computer vision (CV) modules. In some embodiments, the AI and CV modules are supported by a video and AI inferencing pipeline (VAIP) 105. In some embodiments, VAIP 105 supports the AI and CV modules and manages the flow of control and information between the modules. VAIP 105 may comprise a configuration file comprising instructions for connecting and managing the flow. VAIP 105 may support execution of the AI algorithms on a GPU 107. VAIP 105 may also support direct media interfaces (e.g., HDMI, or DVI). One or more outputs of the plurality of inputs processed by the AI pipeline may be generated comprising a landmark location 120 and one or more feature elements identified from the plurality of inputs 109. The one or more outputs may be overlaid onto the video stream input 101 to generate an output 130. In some embodiments, the system 100 comprises a display monitor. In some embodiments, output 130 is displayed on a display monitor (e.g., a monitor, a television (TV)). In some embodiments, the system comprises a displaying device. In some embodiments, landmark location 120 and inputs 109 are sent back to the CCU to be overlaid onto the video stream to generate output 130.

In some embodiments, the arthroscope may generate consecutive images (e.g., a video feed) at a rate of at least about 10 frames per second (fps). In some embodiments, there is a latency in the system, which is the time of required to receive an image (e.g., a video feed) and provide an overlay (e.g., a processed image). In some other cases, two consecutive frames from the video stream (e.g., video stream input 301) may be generated at a speed of 1/fps (frames per second). In some embodiments, the latency in the system is at most 1/fps. The latency of the system may be less than the inverse of the rate of consecutive image generation of the surgical camera. For example, when the input signal is streaming at 20 frames per second, the latency may be equal or less than 1/20 (1/fps) or 50 ms. In some embodiments, the latency may comprise a period of rest in the system.

In some embodiments, the operations may further comprise identifying and labeling one or more elements in the video stream using at least one of a trained computer algorithm, where the one or more elements comprise one or more of an anatomical structure, a surgical tool, an operational procedure or action, or a pathology. In some embodiments, identifying and labeling the one or more elements in the video stream comprises using one or more AI modules. In some embodiments, the one or more AI module may comprise one or more modules for video stream decomposition, tool recognition, anatomy recognition, tool tracking, gesture recognition, landmark point registration, or anatomy and landmark tracking. In some embodiments, the system recommends one or more landmarks based at least partially on the identified elements. In some embodiments, the system recommends one or more landmarks based at least partially on the identified elements.

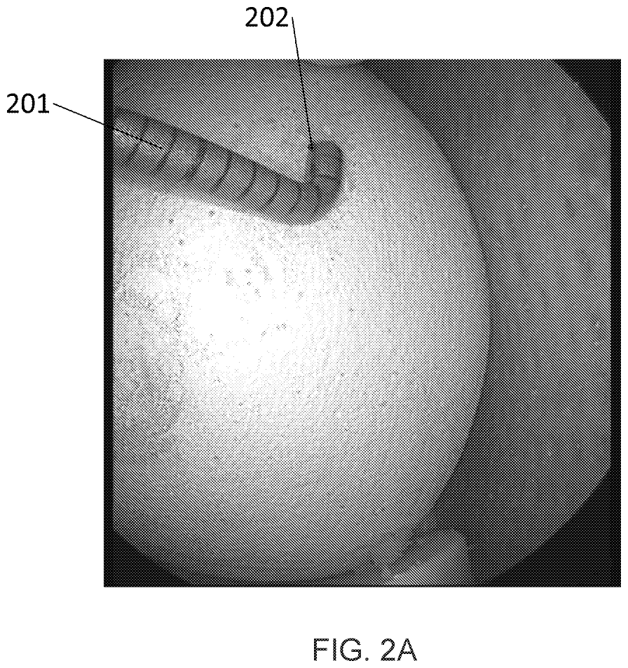

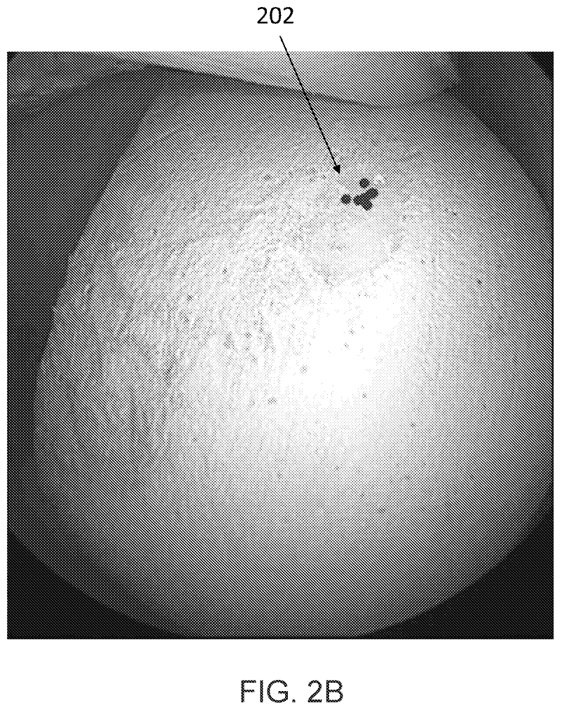

FIG. 2A-2B show examples of landmark placement on a model femoral condyle. In some embodiments, as shown in FIG. 2A, an operator (e.g., a surgeon) indicates a desired location of a landmark using a standard surgical probe 201. The operator may then activate a register command to the system to register the desired location. The location of the landmark can be visualized on the screen displaying the video stream of the surgery with a dot 202 (e.g., a blue dot). In some embodiments, the system saves the location of the landmark and tracks the landmark throughout the surgery. The dot visualizing a landmark can be displayed on the screen or removed from the screen at any moment by the operator. In some embodiments, the landmark is one isolated dot. In some embodiments, landmark is a plurality of isolated dots 202, as shown in FIG. 2B. In some embodiments, the landmark is a virtual arbitrary pattern or a predefined shape. In some embodiments, the location of a landmark can be indicated and/or selected preoperatively. The landmark may be a location of an implant or an anchor location during arthroscopic procedures. In some embodiments, the arthroscopic procedure is used in a rotator cuff repair surgery. In some embodiments, the arthroscopic procedure is used in an ACL repair in a knee surgery. In some embodiments, the arthroscopic procedure is used in a graft placement procedure. In some embodiments, the arthroscopic procedure is used in a decompression procedure. In some embodiments, the decompression procedure comprises removal or reshaping of bony structures to reduce pain. In some embodiments, a shoulder arthroscopic procedure comprises placement of a graft.

In some embodiments, the arthroscopic procedure comprise removal, or resection of an inflamed tissue and/or frayed tendons. In some embodiments, the arthroscopic procedure is used in a removal of or a resection of one or more inflamed tissues. In some embodiments, the arthroscopic procedure is used in a removal of or a resection of one or more frayed tendons where the video stream is monocular. For example, radiological imaging or other imaging methods such as fluoroscopic imaging can be used to locate the place of a landmark. An image obtained from these imaging methods can be provided to the system to be overlaid on the video stream during the surgery. In some embodiments, the system shows a latent anatomical structure or pathology; for example, the operator (e.g., a surgeon) may protect the ureter as the system visualizes the location of the ureter although it may not be exposed during a surgical procedure. For example, the system can ingest information from an external system such as a fluoroscopic imaging system. The landmark may then take the form of the vascularity rendered visible by the fluorescent dye imaged using the fluoroscopic imaging system. The system may continue to retain and track vascularity during the procedure (e.g., arthroscopic surgery).

In some embodiments, the system 100 operates on stored video content. A video recording of an arthroscopic surgery can be played back and sent to an interface. The system may then overlay any landmark on the video stream as explained herein. In some embodiments, the landmark placement on a recording of a surgery is used for training purposes. In some embodiments, the system operates on stereoscopic video streams. In some embodiments, the system can be used during a robotic arthroscopic procedure (e.g., a surgery). In some embodiments, a view of the display may be changed. In some embodiments, by changing a view a landmark that is overlaid on a video stream can be omitted from being displayed. In some embodiments, the operator can select to render the landmark invisible temporarily or throughout the arthroscopic procedure. The operator may revert the view to a previous display. The operator may identify new sets of coordinates for a landmark. The new landmark may be overlaid on to the video stream to be displayed. A plurality of landmarks may be selected to be displayed simultaneously or one at a time. In some embodiments, a change in the view is automatic. In some embodiments, changing a view may be cause by the AI pipeline identifying an anatomical structure or pathology.

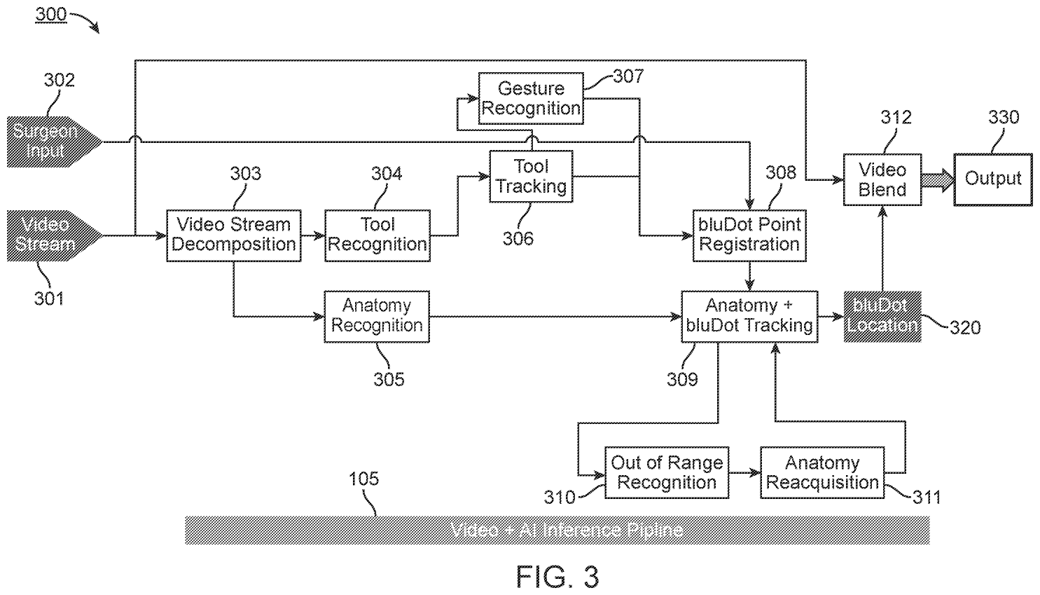

In some embodiments, a set of coordinates of the landmark is provided by an operator intraoperatively. FIG. 3 shows a schematic of an exemplary flow chart of a landmark placement system 300. The system may comprise a plurality of modules which operate on the video stream input 301 generated by an arthroscopic camera and an input received from an operator 302 (e.g., a surgeon). In some embodiments, video stream input 301 is processed by a video stream decomposition module 303 comprising a CV algorithm to decompose a video stream into a series of images. The series of images may be stored in a memory device. One or more images from the series of images may be provided to one or more downstream component comprising a tool recognition module 304 or an anatomy recognition module 305. In some embodiments, video stream decomposition module 303 outputs an image of the field of view of the surgery.

In some embodiments, the tool recognition module 304 uses an AI network to recognize surgical tools in the field of view. Non-limiting examples of the AI network used in tool recognition module 304 may comprise Mask R-CNN, UNET, ResNET, YOLO, YOLO-2, or any combination thereof. In some embodiments, the AI networks are trained to recognize surgical tools of interest using machine learning training comprising architecture-specific training techniques. In some embodiments, the trained AI network detects the presence of a surgical tool in an image and outputs a mask. The mask may be a set of pixels extracted from the input image, which indicate the precise outline of the surgical tool. In some embodiments, the AI network outputs a box (e.g., a rectangular region) in which the tool is detected or displayed.

In some embodiments, the anatomy recognition module 305 uses an AI network to recognize an anatomical structure in a field of view. Non-limiting examples of the AI network used in anatomy recognition module 305 may comprise Mask R-CNN, UNET, ResNET, YOLO, YOLO-2, or any combination thereof. In some embodiments, the AI networks are trained to recognize anatomical structures of interest using architecture-specific training techniques. In some embodiments, the trained AI network recognizes anatomical structures as they are sighted in the field of view. In some embodiments, the trained network outputs pixel masks, which may indicate the precise outline of the recognized anatomical structure. In some embodiments, the trained network outputs a box (e.g., a rectangular region) in which the tool was detected or is displayed.

In some embodiments, an output from tool recognition module 304 is provided to a tool tracking module 306. In some embodiments, the tool tracking module 306 tracks the motion of the one or more tools identified by the tool recognition module 304. In some embodiments, a position of a tool (e.g., an instantaneous position of the tool) may be stored in a memory (e.g., a buffer). In some embodiments, tool tracking module 306 uses CV algorithms to compute the velocity and acceleration of the tool and stores these values in the memory. This data may be stored as a fixed length array. In some embodiments, this array is stored in the time order that they were captured. In some embodiments, the array is stored in descending order of time. The array may have a fixed length and with adding new data, an older entry may be dropped out of the array and the memory buffer. In some embodiments, adding a new entry causes the oldest entry to be dropped out of the array. An output of the tool tracking module 306 may comprise the mask of the recognized tool along with the array of the tool's velocity and/or acceleration. The tool tracking module 306 may supply a position or an array of the positions of one or more tool to a gesture recognition module 307 and a landmark registration module 308 (e.g., a bluDot point registration module).

In some embodiments, the gesture recognition module 307 uses an AI network comprising a memory (e.g., a recurrent neural network (RNN)), to interpret the movement of the tools. In some embodiments, the AI network is trained to recognize specific tools and/or identify specific movement patterns. For example, a tap would involve the tool moving in a specific manner relative to the background anatomy. In some embodiments, the surgeon can indicate a position of an arbitrary landmark by using a predetermined gesture using a surgical tool. Non-limiting example of a gesture may comprise tapping, double tapping, triple tapping, wagging (e.g., moving a tool from left to right). In some embodiments, gesture recognition module 307 outputs a label of the gesture made by an operator using a tool. In some embodiments, gesture recognition module 307 recognizes a gesture made by the operator and generates a label of the name of the recognized gesture to be supplied to a downstream component, which may be landmark registration module 308.

In some embodiments, the landmark registration module 308 receives one or more inputs from tool tracking module 306 and/or gesture recognition module 307, as described herein. In some embodiments, the input from gesture recognition module 307 instructs landmark registration module 308 that a gesture from an operator is recognized. The gesture may then be mapped to an action. In some embodiments, the mapping is configured preoperatively and is loaded from a database when the system is initialized. Non-limiting examples of an action mapped by 3 landmark registration module 08 may comprise to initiate, to replace, to clear all. In some embodiments, landmark registration module 308 may be initiated to assign a unique identifier to a landmark (e.g., a bluDot). An action comprising a command to clear one or more landmarks or clear all may activate landmark registration module 308 to update a list of one or more landmarks. An initiate action may trigger landmark registration module 308 to supply the location of the tool to anatomy and landmark tracking component 309. A replace action may trigger landmark registration module 308 to replace the data associated with the location of one or more landmarks with a location of a new landmark. A Clear all action may trigger landmark registration module 308 to clear any landmark that is being displayed or is stored in the memory. In some embodiments, landmark registration module 308 receives a direct input from the operator to place, replace, or clear a landmark. The direct input may be provided using a digital or mechanical button, for example, a foot-pedal-press or the press of a dedicated button on the arthroscopic device. In some embodiments, the CCU communicates the direct input through a custom interface to the VAIP, described herein. For example, a custom interface may comprise a gesture mapped to an action that is customized for an operator.

In some embodiments, landmark registration module 308 makes a distinction between the manner in which the landmarks are specified (e.g., preoperatively, intraoperatively via a gesture, intraoperatively via direct command from an operator) for rendering and/or recall purposes. For example, a landmark obtained from preoperative planning would be saved from deletion. In some embodiments, landmark registration module 308 supplies a set of coordinates of a tool identified by tool recognition module 304 in the image of the surgical field of view. The updated list in landmark registration module 308 may be passed down to a downstream anatomy and landmark tracking component 309, which may stop tracking and displaying landmarks that may be set for being cleared. For example, fluorescent dyes may be injected in the blood vessels to assist an operator (e.g., a surgeon) in identifying an artery or a vein. In some embodiments, the surgery is performed in close proximity to highly vascular regions, where nicking an artery or a vein can have serious consequences. An image of the identified arteries or veins (e.g., by using a dye) may be received and overlaid on the surgical video by VAIP as a landmark. A confidence may be calculated, substantially continuously, representing a certainty in the accuracy of VAIP to track and recall the landmark (e.g., identified arteries or veins). In some embodiments, the confidence may lower than a threshold, where the threshold may be about 80%, 70%, 60%, 50%, 40%, 30%, 20%, 10% or lower. The threshold may be set by the operator. In some embodiments, the change in confidence is due to changes in the surgical field of view (e.g., operation being performed may change the anatomical structure). The system may then indicate that the confidence in tracking the landmark has diminished. The operator may feature the landmark by, for example, injecting dyes into the blood vessels. The system may then replace the previously identified landmark with newly identified landmark and overlay the landmark on the surgical image (e.g., video stream).

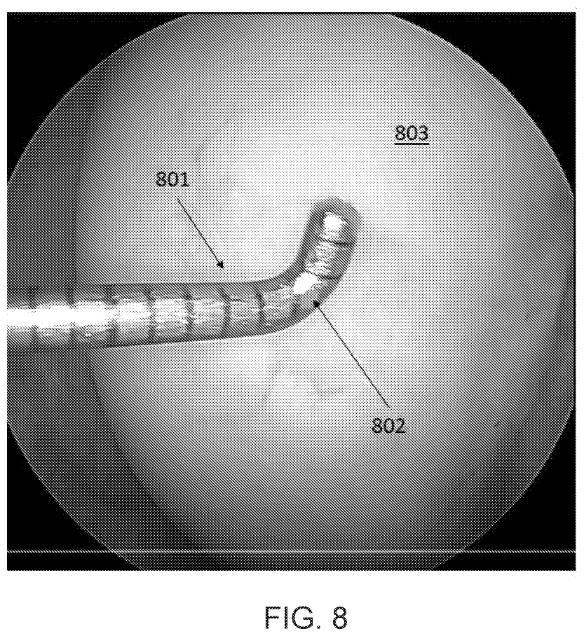

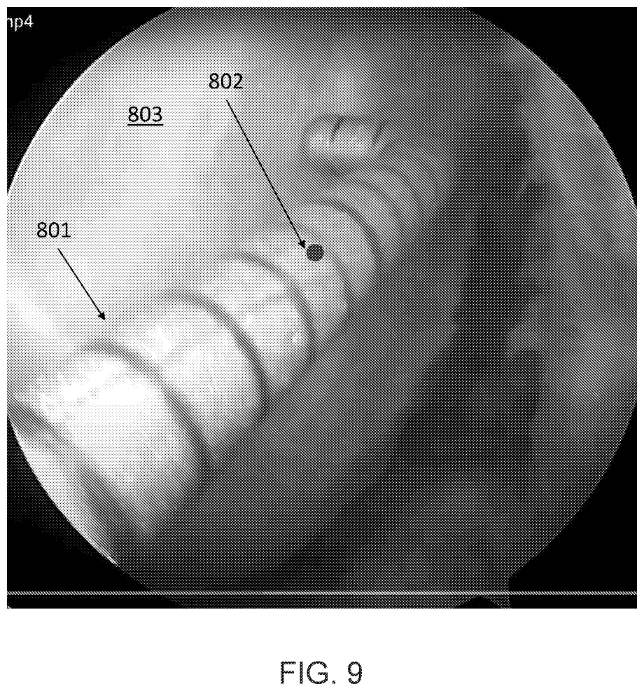

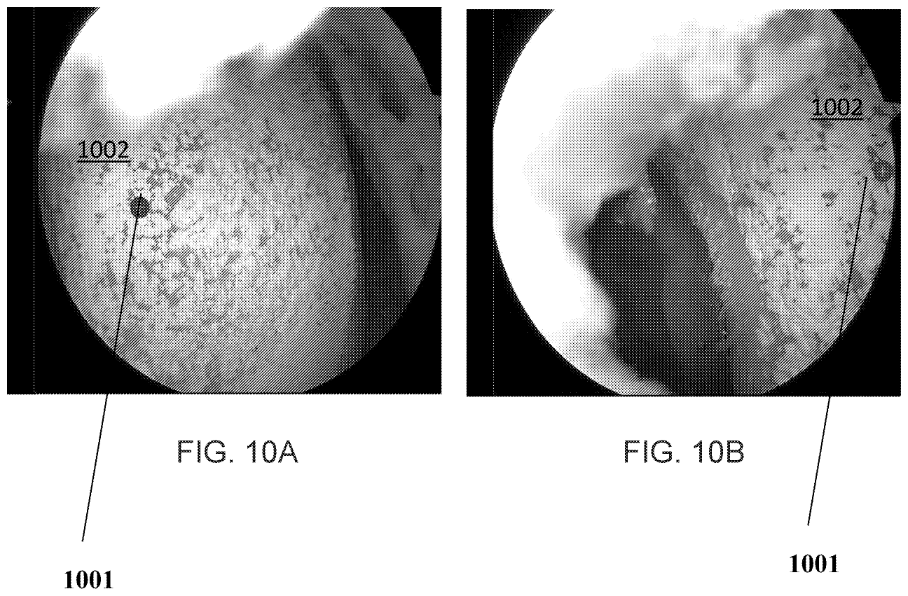

In some embodiments, the anatomy and landmark tracking component 309 receives an anatomy mask from anatomy recognition module 305 and/or an identified tool mask from tool recognition module 304 in a substantially continuous manner. In some embodiments, anatomy and landmark tracking component 309 receives an input from landmark registration module 308 that indicates an action, anatomy and landmark tracking component 309 performs a series of operations. The series of operations may comprise determining the superposition of the tool and the anatomical structure from the mask to determine over which anatomical structure the tool is placed or held. The coordinates of the tool and the anatomical structure may be used to identify the overlap of the tool with the anatomical structure. The series of operations may further comprise extracting a feature to locate a landmark (e.g., a bluDot) in relation to a location of one or more anatomical structures. In some embodiments, the feature comprises a detail on the image, which may comprise a pattern of vascularity, an edge of tissue, or a locally unique patch of pixels. Using the feature, the system may stabilize the landmark (e.g., bluDot) against a movement of the camera with respect to an anatomical structure (also shown in FIGS. 10A-10B). In some embodiments, the feature may comprise points on the tool in the surgical field of view, where the tool is moving independent of an anatomical structure. The feature on the tool may then be excluded in anatomy and landmark tracking component 309 to stabilize the landmark against movements of the tool (also shown in FIGS. 8 and 9).

In some embodiments, the landmark is initialized and designated at an initial position of the tool. In some embodiments, anatomy and landmark tracking component 309 identifies changes in a location of a feature on an anatomical structure and re-designate the location of the landmark. In some embodiments, anatomical structures are modeled as mildly deformable solids and suitable computational techniques are used to track the features on the anatomical structures. In some embodiments, anatomy and landmark tracking component 309 acquires a feature continuously and tracks a movement of the landmark on an expanded canvas. An expanded canvas may comprise the surgical field of views in different images acquired from the video stream that may be connected to one another to generate a larger field of view. In some embodiments, using the feature described herein, the system tracks the landmark with a high degree of certainty even if the landmark or the underlying anatomical structure move off the camera's field of view. In some embodiments, during surgery, the operator might move the camera away from the location of the landmark and surrounding tissues causing the operator to lose sight of the landmarks. In some embodiments, the location of the landmark needs to be re-acquire when the operator returns to the general area again. In some case, an anatomical structure is recognized first, as described before, excluding any tools in the field of view. One or more features may be identified to recognize the location of the landmark according to an anatomical structure that has been recognized before.

For example, one or more feature points may be identified that may be separated by the anatomical structures on which they appear. When the surgeon reenters a surgical field of view that has been analyzed in a previous image, the anatomical recognition module(s) may recognize the previously processed image. Upon matching the new coordinates of the feature points in the current image with the coordinates of the feature points in the previously processed image, the landmark may be placed in its location. The location of the landmark may be reestablished based on the feature points in the current image as well as the previously identified feature points. The feature points matching process may be repeated to increase the accuracy of landmark placement. This process may be performed using parallel computing (e.g., a GPU). The system may discard the feature points identified in the previously processed image and replace them with the feature points identified in the current image. The processed described herein may be performed using the anatomy and landmark tracking module 309, out of range recognition module 310, and anatomy reacquisition module 311.

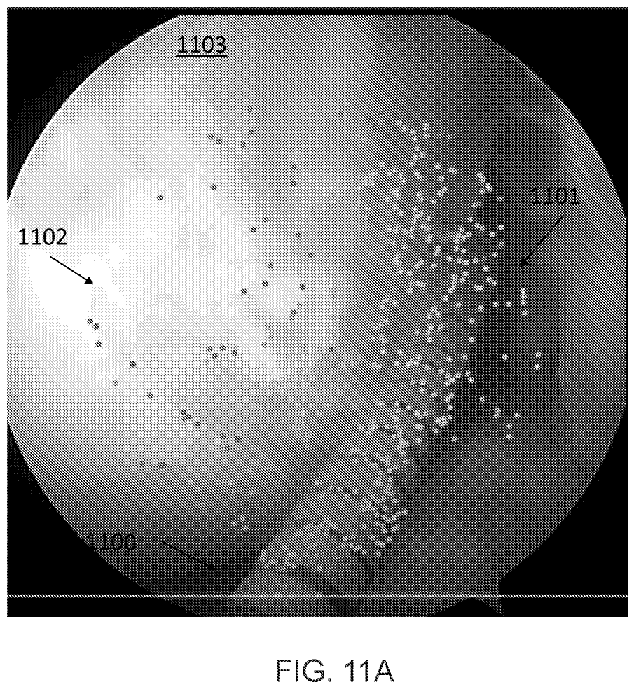

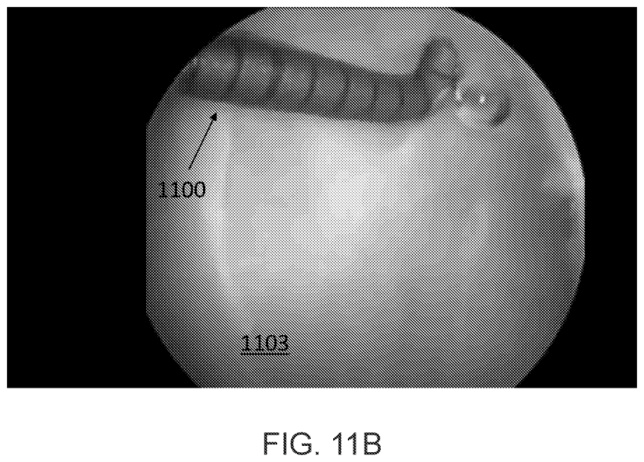

FIGS. 11A-11B shows an example of feature detection. For example, a plurality of features (or feature points) 1101 may be detected on a tool 1100 (shown as green points 1101 in FIGS. 11A-11B) may be distinguished from set of features 1102 recognized on the anatomical structure 1103 (shown as red points 1102 in FIGS. 11A-11B). In some embodiments, during the surgical procedures the surgical field of view may be altered. For example, a procedure may comprise debridement of soft tissue that may change the field of view. Once the tool is recognized and tracked (e.g., in real time), the feature points detected on the tool may be eliminated. In some embodiments, the feature points detected on the anatomical structure may be used to track the landmark. This may improve stabilize a landmark against tool movements that may block a landmark. FIG. 8 shows an example of occlusion by the tool being ignored, when overlaying landmark on a video or image. In some embodiments, bleeding or body fluids may change the field of view. In some embodiments, the operations in anatomy and landmark tracking component 309 comprise continuously acquiring features from the field of view and discarding features that are missing in consecutive images to stabilize the landmark against the changes in the field of view from an action being performed in a procedure. The features may be acquired against an anatomical structure as reference. In some embodiments, anatomy and landmark tracking component 309 comprises an out of range recognition module 310 and an anatomy reacquisition module 311. In some embodiments, anatomy and landmark tracking component 309 updates the location of the landmark based on a feature in the observable portion of an anatomical structure. In some embodiments, as described herein, the field of view may be shifted excluding the anatomical structure or the landmark. As the camera pans back to the location of the landmark, anatomy and landmark tracking component 309 may increase the confidence of the position of the landmark by using out of range recognition 310 and and anatomy reacquisition module 311, as described herein. The output of anatomy and landmark tracking component 309 comprise a location of the landmark (e.g., bluDot) in the field of view and/or within a frame or boundaries of an image being processed. The location of the landmark is sent to the module 320. The landmark may then be overlaid on the output video stream 330. An example of the output video stream 330 is shown in FIG. 8. In some embodiments, the surgical field of view is about 3 centimeters (cm) to about 6 cm. In some embodiments, the surgical camera's (e.g., arthroscope) range of movement is about 3 cm to about 6 cm. In some embodiments, the range for stabilization is similar to the surgical camera's range of movement, which is about 3 cm to about 6 cm. in some embodiments, the precision in stabilizing the landmark against the changes in the field of view is about 1 millimeters (mm) to about 3 mm.

In some embodiments, the output from the landmark location module 320 is overlaid onto the video stream input from module 301 in a video blend module 312. The output from video blend module 312 may be displayed on output video stream 330 (e.g., with a screen, a monitor, a TV, a laptop screen, etc.) The output from 320 may be directed to the camera control unit to be scaled and overlaid onto the video stream of the procedure.

Another aspect of the invention provides a system for assisting an arthroscopic procedure by allowing computer-implemented arbitrary landmark placement using radiological imaging. The system may comprise one or more computer processors and one or more non-transitory computer-readable storage media storing instructions that are operable, when executed by the one or more computer processors, to cause the one or more computer processors to perform operations comprising: receiving a radiological image of a subject; generating a 3D representation of the radiological image; identifying an anatomical structure in the radiological image with a trained machine learning algorithm; receiving a location of a landmark from an operator; overlaying the landmark on the 3D representation of the radiological image; and displaying the overlay on a displaying device to be used by the operator.

In some embodiments, an operator identifies or set a location of a landmark during a preoperative surgery planning phase. In some embodiments, the landmark may be set by an operator (e.g., a surgeon) on a radiology images obtained from a subject. The landmark may then be supplied to a landmark registration module similar to landmark registration module 308 in FIG. 3. This may allow the operator to hide or display the landmark during the preoperative surgery planning phase.

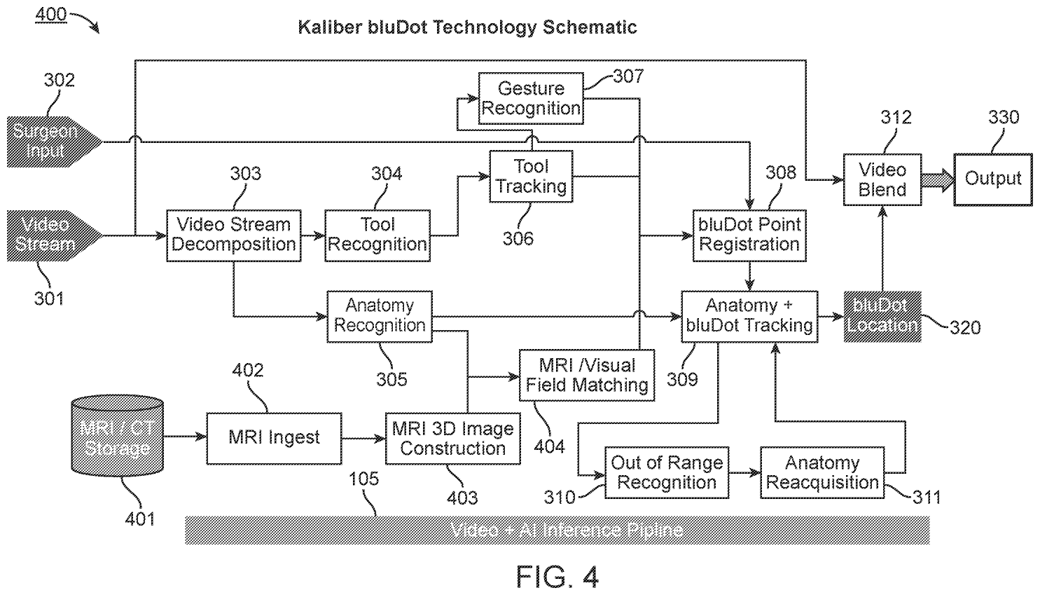

FIG. 4 shows a schematic of an exemplary workflow of landmark placement using a radiology imaging. As shown in FIG. 4 a plurality of modules may be added to the system 300 shown in FIG. 3 to allow using preoperative medical imaging data of a subject 401 (e.g., radiology imaging data such as MRI or CT scan) to set a location of a landmark on a video stream of an arthroscopic procedure (e.g., from video stream input 301). In some embodiments, a preoperative medical imaging ingest module 402 interfaces with an external repository to import the preoperative medical imaging data 401. The preoperative medical imaging data may comprise radiological images of a subject. In some embodiments, the radiological images are from and associated with a joint or other boney structure of the subject such as the shoulder, knee, hip, ankle or spine. In various embodiments, the radiological images may be generated using one or more of fluoroscopy, magnetic resonance imaging (MRI), X-ray, computed tomography (CT) scanning, positron emission tomography (PET) scanning or ultrasound. In some embodiments, the preoperative medical images comprise In some embodiments, MRI or CT scan images is acquired from the subject for an arthroscopic procedure (e.g., a knee surgery, a shoulder surgery, or a hip surgery). The MRI or CT scan images may comprise an image of a subject's knee or a shoulder. In some embodiments, the MRI, CT scan or other images are obtained from the repository in a standard format (e.g., DICOM). In some embodiments, preoperative medical imaging ingest module 402 comprises an application programming interface (API) layer to abstract external system associated with an imaging system (e.g., MRI, CT scan or PET imaging) from the system 400. In some embodiments, the repository of images comprises an image of a landmark. In some embodiments, the image of the landmark from the repository has been placed by an operator (e.g., a surgeon) on the MRI or CT scan images of the subject. The output from preoperative medical imaging ingest module 402 may be provided to a three dimensional (3D) image reconstruction module 403. In some embodiments, 3D image reconstruction module 403 converts volumetric data in images from preoperative medical imaging ingest module 402 comprising one or more slices of two dimensional (2D) images and converts the data into a 3D image in a computer memory. In some embodiments, the coordinates of the landmarked set by the operator are mapped onto the 3D image. In some embodiments, 3D image reconstruction module 403 may generate a multi-dimensional array comprising the 3D representation of a radiological image and the landmark mapped to the image. In some embodiments, the output from 3D image reconstruction module 403 may be merged with the mask(s) generated by the anatomy recognition module 305, using a mapping module 404. In some embodiments, 404 comprises a trained AI network to recognize anatomical structures in an image obtained preoperatively (e.g., an MRI or a CT scan image). In some embodiments, the anatomical structure may comprise a bony structure. In some embodiments, the anatomical structure may comprise a tendon. In some embodiments, the anatomical structure recognized in the image (e.g., an MRI or a CT scan image) may be masked (e.g., labeled) in mapping module 404 using the same labeling system used in anatomy recognition module 305. The anatomical structure recognized in mapping module 404 may then be matched to an anatomical structure recognized in anatomy recognition module 305. In some embodiments, the landmark specified in 3D image reconstruction module 403 may be mapped onto the anatomical structure recognized in anatomy recognition module 305. The mapping may be provided to landmark registration module 308. As described hereinbefore, landmark registration module 308 may process and send the landmark and the anatomical structure information to be overlaid onto the video stream of the surgery. In some embodiments, 320 is adjusted for the movement of the surgical camera. In some embodiments, when a similar structure is identified from a preoperative medical image (e.g., MRI or CT scan image) and from an image from a vide stream of the surgery, the two anatomical structures are matched (e.g., in mapping module 404), which corrects the frame for any image discrepancies associated with the surgical camera movement. In some embodiments, each frame from the video stream is corrected for the movement of the surgical camera.

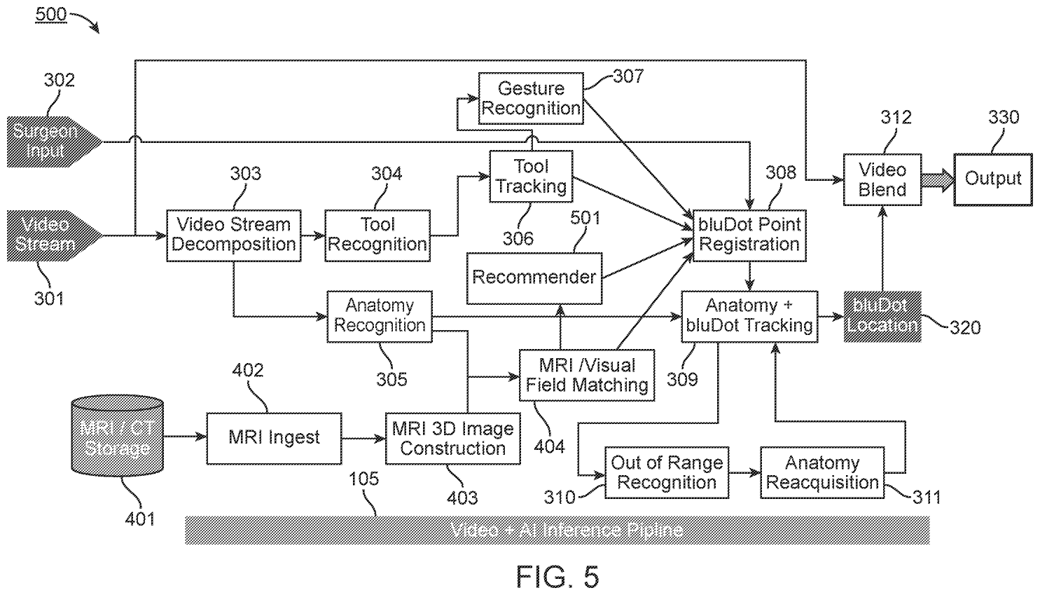

In some embodiments, the system comprises a recommender module that can recommend placement of a landmark based at least in part on the surgical context. FIG. 5 shows a schematic of an exemplary workflow of a system to recommend a landmark placement. The system 500 shown in FIG. 5 may comprise the system 400 and a recommender module 501 to make the recommendation for placing a landmark. In some embodiments, system 500 is a surgical decision support system. In some embodiments, 501 receives anatomical feature masks or anatomical structure masks from mapping module 404. In some embodiments, based on the anatomical feature masks or anatomical structure masks received, 501 identifies a context of the surgery (e.g., anatomical region or a portal).

In some embodiments, based at least on the identified context 501 recommends the placement of a landmark. Non-limiting examples of recommendations from 501 may comprise a femoral and tibial tunnel placement in an anterior cruciate ligament (ACL) surgery, or an anchor placement in a Rotator Cuff Repair surgery. In some embodiments, 501 recommends the location of a landmark based at least in part on the location of the landmark in a preoperative medical image of the subject identified by 3D image reconstruction module 403 and/or mapping module 404. The recommended landmark and/or landmark location may be sent to landmark registration module 308 to be processed, overlaid on the video stream of the surgery and to be displayed on a displaying device (e.g., a monitor), as described herein before. In some embodiments, a preoperatively acquired image (e.g., MRI, CT scan, etc.) may be processed as described herein combined with tool tracking module 306 to provide the information required to estimate a size or location of a landmark. For example, imaging modalities (e.g., CT Scan, MRI) may produce images containing anatomical features that can be recognized by the system as described herein. The images may further comprise a location of a landmark. In some embodiments, these images are three dimensional images comprising voxels, where a voxel, (volumetric pixel), can represent a volume in physical space. Therefore, a location of a landmark may be identified on a surgical field of view image by matching the identified anatomical structures (e.g., by recognizing anatomical features on a preoperative image and the surgical image). The location of the landmark may be further identified based in part by measuring a size of the anatomical structure based in part on the size of the voxels in the preoperative image. This measurement may be used to place the landmark on a location on the anatomical structure on the surgical image corresponding to the location of the landmark identified on the preoperative image (e.g., CT scan, MRI).

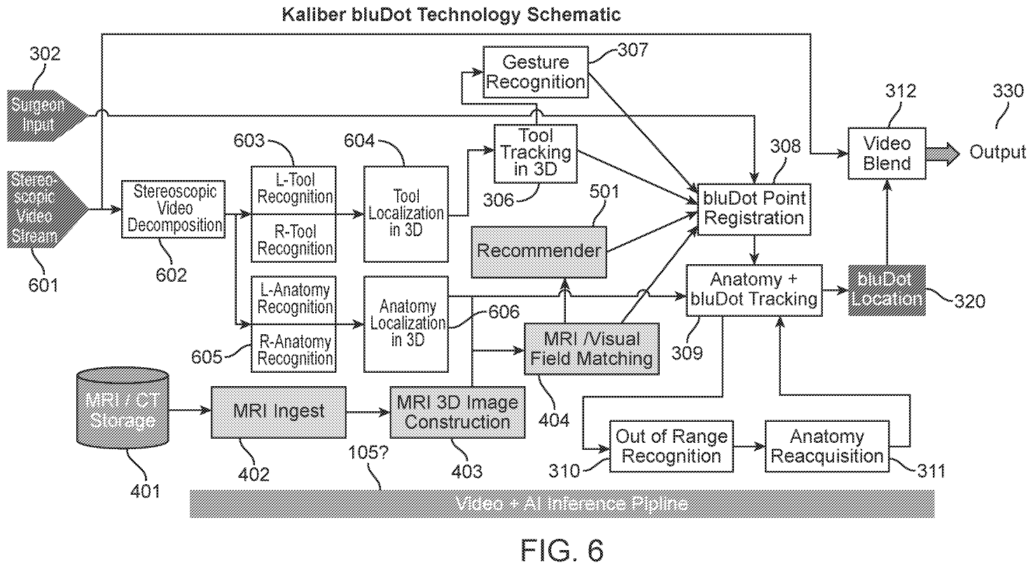

In some embodiments, the system is configured to process a video stream from a stereoscopic surgical camera (e.g., a stereoscopic arthroscope). FIG. 6 shows a schematic flowchart of an exemplary system to process a stereoscopic video stream (e.g., a 3D video). In some embodiments, the system 600 comprises the components in system 500 and a plurality of modules to process stereoscopic video input or stream 601. In some embodiments, the stereoscopic video input or stream 601 is first processed by a stereoscopic video decomposition module 602 to generate an image from the stereoscopic video input or stream 601. In some embodiments, stereoscopic video decomposition module 602 provides an image from the stereoscopic video input or stream 601 to a tool recognition module 603 and/or an anatomy recognition module 605. The modules tool recognition module 603 and anatomy recognition module 605 are similar to tool recognition module 304 and anatomy recognition module 305, respectively. In some embodiments, tool recognition module 603 and anatomy recognition module 605 are capable of processing a surgical field in an image that has a shifted view due to parallax in a stereoscopic video stream. A stereoscopic video stream or an image from the stereoscopic video stream may comprise two channels (e.g., a right side, a left side). The parallax may comprise a displacement or difference in the apparent position of an object viewed along two different lines of sight. In some embodiments, tool recognition module 603 provides one or more masks for a surgical tool to a tool localization module tool localization module 604. In some embodiments, tool recognition module 603 provide one or more masks for an anatomical structure to an anatomy localization module 606. In some embodiments, tool localization module 604 uses the differences in the perspectives of a given tool and localizes the tool in 3D space. In some embodiments, tool localization module 604 comprises tool recognition algorithms which are applied to the two channels of an image from a stereoscopic video stream (e.g., binocular video stream). A landmark may be registered using a surgical tool, as mentioned herein. In some embodiments, the landmark appears in 3D space when viewed using a 3D viewing device (e.g., a binocular viewer). In some embodiments, anatomy recognition module 605 provides one or more masks for an anatomical structure to an anatomical localization module 606. In some embodiments, anatomy localization module 606 processes the anatomical structure mask(s) in the two channels of the image from a stereoscopic video stream and generates a mask that can be visualized in a 3D viewer based on the spatial information of the anatomical structure provide by anatomy recognition module 605. In some embodiments, a landmark (e.g., a bluDot) is rendered to the field of view in a way that the landmark is placed in a left and a right channel of stereo display channels independently. In some embodiments, the landmark is placed with a shift (e.g., laterally) to generate depth in perception. The output video stream 330 may comprise a landmark visualized or displayed in 3D overlaid (e.g., attached) onto an anatomical structure in a video stream of a surgery.

In some embodiments, an object (e.g., a probe or surgical tool) 801 may be placed at the same location of a landmark 802. The system may identify the tool 801 as described herein and compensate for any occlusions (FIG. 8). The landmark 802 may also move corresponding to its location as an anatomy 803 moves. FIG. 9 shows another example of the landmark 802 being cleared from an object (e.g., a tool) 801 that could block the landmark 802. FIG. 10A and FIG. 10B show the performance of the system in stabilizing a landmark 1001 (e.g., bluDot) against a movement of a camera with respect to an anatomical structure. The camera may move with respect to an anatomical structure 1002, but a landmark 1001 may remain in a marked location on the anatomical structure. In other words, the landmark 1001 may move with the anatomical structure 1002 as camera moves around.

### Computer Systems

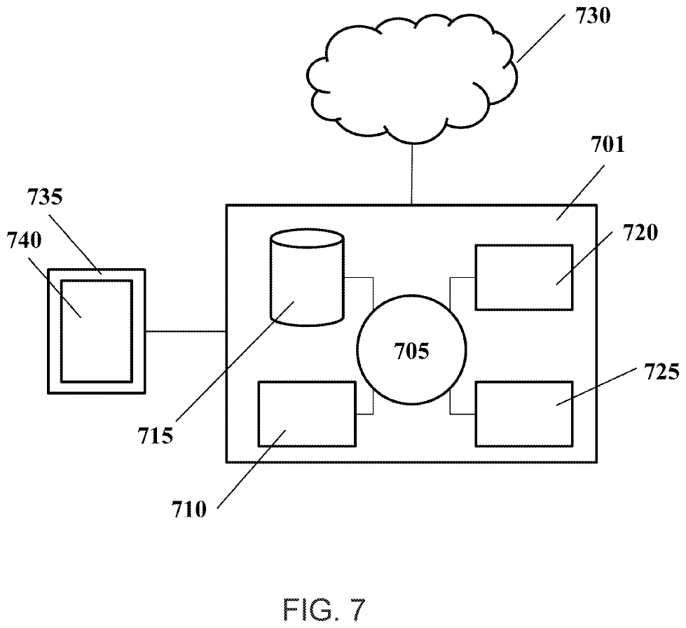

Various embodiments of the invention also provide computer systems that are programmed to implement methods of the invention. Accordingly, a description of one or more embodiments of such computer systems will now be described. FIG. 7 shows a computer system 701 that is programmed or otherwise configured to perform one or more functions or operations of methods of the present invention. The computer system 701 can regulate various aspects of the present invention, such as, for example, of receiving an image from an interventional imaging device, identifying features in the image using an image recognition algorithm, overlaying the features on a video feed on a display device, make recommendations or suggestion to an operator based on the identified features in the image. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some embodiments is a telecommunication and/or data network. The network 730 can include one or more computer servers, which can enable distributed computing, such as cloud computing. The network 730, in some embodiments with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present invention. Examples of operations performed by the CPU 705 can include fetch, decode, execute, and writeback.

The CPU 705 can be part of a circuit, such as an integrated circuit. One or more other components of the system 701 can be included in the circuit. In some embodiments, the circuit is an application specific integrated circuit (ASIC).

The storage unit 715 can store files, such as drivers, libraries and saved programs. The storage unit 715 can store user data, e.g., user preferences and user programs. The computer system 701 in some embodiments can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a portable computer, a tablet, a smart display device, a smart tv, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some embodiments, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. In various embodiments machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks (including wireless and wired networks). Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, an overlay of the identified features on a video feed from an arthroscope or to provide a recommendation to an operator in the course of a surgery. Examples of UI's include, without limitation, a graphical user interface (GUI) and web-based user interface.

In various embodiments, the methods and systems of the present invention can be implemented by way of one or more algorithms. An algorithm can be implemented by way of software upon execution by the central processing unit 705. The algorithm can, for example, receiving an image from an interventional imaging device, identifying a feature in the image using an image recognition algorithm, overlaying the features on a video feed on a display device, make recommendations or suggestion to an operator based on the identified feature in the image.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. Accordingly, it should be understood that the invention covers various alternatives, modifications, variations or equivalents to the embodiments of the invention described herein.

Also, elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Further, embodiments of the invention specifically contemplate the exclusion of an element, act, or characteristic, etc. when that element, act or characteristic is positively recited. Hence, the scope of the present invention is not limited to the specifics of the described embodiments but is instead limited solely by the appended claims.

1. A system for assisting a minimally invasive procedure by allowing computer-implemented arbitrary landmark placement, the system comprising one or more computer processors and one or more non-transitory computer-readable storage media storing instructions that are operable, when executed by said one or more computer processors, to cause said one or more computer processors to perform operations comprising:
receiving a video stream from an arthroscopic imaging device;
receiving one or more sets of coordinates of one or more landmarks;
overlaying said one or more landmarks on said video stream; and
displaying said overlay on one or more display devices intraoperatively to be used by an operator during said arthroscopic procedure.

2. (canceled)

3. The system of claim 1, wherein said operations further comprise identifying and labeling one or more elements in said video stream using at least one of a trained computer algorithm and one or more modules, wherein said one or more elements comprise one or more of an anatomical structure, a surgical tool, an operational procedure or action, or a pathology.

4. (canceled)

5. The system of claim 3, wherein said one or more modules comprise video stream decomposition, tool recognition, anatomy recognition, tool tracking, gesture recognition, landmark point registration, or anatomy and landmark tracking.

6. The system of claim 3, wherein said system recommends one or more landmarks based at least partially on said identified elements.

7. The system of claim 1, wherein said operations further comprise: storing the one or more sets of coordinates of one or more landmarks; changing a view of said display to omit said overlaid landmark from being displayed; reverting said view to said previous display; identifying said one or more set of coordinates for said one or more landmarks; and re-overlaying said one or more landmarks.

8. The system of claim 7, wherein said operator activates said changing and said reverting steps.

9. The system of claim 7, wherein said changing a view step is activated automatically based on a change in an identified anatomical structure or pathology.

10. (canceled)

11. (canceled)

12. The system of claim 1, wherein said one or more sets of coordinates of said one or more landmarks is generated from one or more medical images of a subject.

13. (canceled)

14. (canceled)

15. The system of claim 1, wherein said radiological images are associated with a shoulder, a knee, or a hip of said subject.

16. (canceled)

17. The system of claim 1, wherein said video stream is provided by an arthroscope during an arthroscopic procedure.

18. (canceled)

19. (canceled)

20. (canceled)

21. (canceled)

22. (canceled)

23. (canceled)

24. (canceled)

25. The system of claim 1, wherein said one or more computer processors receive said video stream from one or more camera control units using a wired media connection.

26. The system of claim 25, wherein a latency between receiving said video stream from the one or more camera control units and overlaying said output and said videos stream is at most 40 milliseconds (ms) to accommodate a digital camera with about 24 frames per second (fps).

27. The system of claim 25, wherein a latency between receiving said video stream from the one or more camera control units and overlaying said output and said videos stream is no more than a time between two consecutive frames from said digital camera.

28. The system of claim 1, wherein said one or more computer processors receive said video stream from one or more camera control units using a network connection.

29. (canceled)

30. (canceled)

31. The system of claim 1, further comprising a camera control unit configured to control a light source, capture digital information produced by said digital camera.

32. The system of claim 31, wherein said camera control unit converts said digital information produced by said digital camera into said video stream.

33. The system of claim 31, wherein said camera control unit record said digital information produced by said digital camera in a memory device.

34. (canceled)

35. (canceled)

36. (canceled)

37. The system of claim 31, wherein said camera control unit is configured to overlay said output from with said one or more computer processors with said video stream.

38. (canceled)

39. (canceled)

40. (canceled)

41. The system of claim 1, further comprising an input, wherein said input comprises one or more of: a push-button, a touchscreen device, a foot pedal, a gesture recognition system, or a voice recognition system.

42. (canceled)

43. The system of claim 42, wherein said one or more landmarks are tracked during said minimally invasive procedure, further wherein said tracking of one or more landmarks is associated with said set of coordinates of said one or more landmarks relative to at least an anatomical structure.

44-112. (canceled)
